(19) United States
(12) Patent Application Publication
(10) Pub. No.: US 2008/0223116 A1
(43) Pub. Date: Sep. 18, 2008

(54) Drug Release Cell and a Method for Testing the Drug Release of a Suspension in a Liquid Utilizing the Same

(76) Inventors: Emad Alkhawam, New City, NY (US); Mankit Ho, West Nyack, NY (US); Jin Qiang Xia, Ridgefield, NJ (US); Satheesh Papineni, Harriman, NY (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/685,520

(22) Filed: Mar. 13, 2007

(51) Int. Cl. G01N 13/00; G01N 13/04; G01N 33/15

(52) U.S. Cl. 73/64.56; 73/64.47; 73/866

(57) ABSTRACT

A drug release cell useful in testing the drug release of a product in a liquid includes a first chamber and a second chamber with a membrane positioned in between such that the first chamber is located above the membrane and the second chamber is located below the membrane. The membrane has pores sized to permit a liquid and a released product, such as an insulin suspension, disposed in the second chamber to pass therethrough. When the drug release cell is filled with liquid and product is present in the second chamber the liquid is mixed with a mixing device to circulate the liquid and disperse released media throughout the liquid. Samples are taken at predetermined times from the first chamber through a sampling port to determine the concentration of the product in the liquid. The concentration data may then be utilized to create a drug release profile. Different formulations of a product may be tested in the drug release cell and the concentration data may be utilized to create distinguishable drug release profiles for each formulation.

### BACKGROUND OF THE INVENTION

1. Field of the Invention

Disclosed herein is a drug release cell and a method for testing the drug release of a product in a liquid.

2. Background Art

Before a drug can enter the market it is necessary to determine the rate at which the drug will pass through various barriers of the body. For example, ingestible drugs will need to pass through the intestinal walls to enter the bloodstream and transdermal drugs will need to pass through the skin to enter the bloodstream. The rate at which the drug will pass through the specific barrier of the body may be determined through the utilization of a variety of apparatuses that simulate the human condition, utilizing for example a filter, mesh screen, or membrane that acts like the actual biological barrier.

A number of apparatuses exist for determining the dissolution rate of a solid drug dosage. One example is the basket method, disclosed in the United States Pharmacopeia (USP 29) on pages 2673-2674 and 2675-2676, wherein a cylindrical basket having side walls made of a mesh material holds a solid drug dosage. A cover is placed on the top of the cylindrical basket with a stirring shaft attached thereto. The cylindrical basket is immersed in a vessel containing a liquid bath and the liquid flows into the interior of the cylindrical basket. The stirring shaft is then rotated to spin the cylindrical basket such that the solid drug dosage dissolves in the liquid. The dissolved drug dosage then passes through the mesh of the cylindrical basket into the vessel. Samples are taken from the vessel at specified time intervals to determine the drug release rate.

A similar example is the paddle method, disclosed in the United States Pharmacopeia (USP 29) on pages 2674-2677, which has the same assembly as the basket method except the stirring shaft extends into the cylindrical basket and has a paddle at its end.

Yet another example for determining the rate at which a solid drug dosage will pass through a barrier is the continuous flow-through cell. An example is disclosed in the United States Pharmacopeia (USP 29) on pages 2678-2679. A solid oral dosage form is placed in a flow-through cell that has a filter or screen attached to one end and a second end that is open but blocked by at least one glass bead. The flow-through cell is placed in a vessel containing a liquid bath. A pump forces the liquid through the open end of the flow-through cell past the glass bead and out the end with the filter attached thereto. The pumping of the liquid through the flow-through cell stirs the liquid in the cell, causing the solid drug dosage to dissolve in the liquid. The dissolved drug dosage then passes through the filter and samples are collected at specified time intervals to determine the drug release rate.

An apparatus, commonly referred to as a Franz cell, is known for determining the rate at which a cream-based drug will pass through a barrier. An example is disclosed in U.S. Pat. No. 5,296,139. In this device, a cell is divided into a first chamber and a second chamber by a membrane. A sample is placed on top of the membrane in the first or upper chamber. The second or lower chamber has a liquid inlet tube and a sample outlet tube and a spring or coil. A liquid is introduced into the cell through the inlet tube in an amount to fill the second chamber. The spring rotates to stir the liquid causing the sample to dissolve in the liquid and pass through the membrane into the second chamber. Samples are taken from the sample tube located in the second chamber at specified time intervals to determine the rate at which the sample has passed through the membrane.

### BRIEF SUMMARY OF THE INVENTION

Disclosed herein is a drug release cell having a first vessel having a first chamber; a second vessel having a second chamber; a membrane positioned between said first vessel and said second vessel such that said first chamber is located above said membrane and said second chamber is located below said membrane, wherein said membrane has pores sized to permit a liquid and a released product disposed in said second chamber to pass therethrough; a sample port connected to said first chamber; and a mixing device located in said second chamber.

Also disclosed herein is a method for testing the drug release of a product in a liquid including providing a drug release cell having a first chamber; a second vessel having a second chamber; a membrane positioned between said first vessel and said second vessel such that said first chamber is located above said membrane and said second chamber is located below said membrane, wherein said membrane has pores sized to permit a liquid and a released product disposed in said second chamber to pass therethrough; a sample port connected to said first chamber; and a mixing device located in said second chamber. Product is placed in said second chamber, the drug release cell is filled with said liquid; and said liquid is mixed utilizing said mixing device to circulate said liquid and evenly disperse said product throughout said liquid as it releases.

In addition, disclosed herein is a method for testing the release of a suspension in a liquid including providing a drug release cell having a first chamber; a second vessel having a second chamber; a membrane positioned between said first vessel and said second vessel such that said first chamber is located above said membrane and said second chamber is located below said membrane, wherein said membrane has pores sized to permit a liquid and a released product disposed in said second chamber to pass therethrough; a sample port connected to said first chamber; and a mixing device located in said second chamber. Product is placed in said second chamber, the drug release cell is filled with said liquid; and the drug release cell gives distinguishable drug release profiles for suspensions having different rates of drug release.

### BRIEF DESCRIPTION OF THE DRAWINGS/FIGURES

FIG. 1 is an exploded view of an exemplary drug release cell.

FIG. 2 is a plot of the drug release profiles for various insulin formulations using data taken with the exemplary drug release cell of FIG. 1.

FIG. 3 is a plot of the drug release profiles for various insulin formulations using data taken with the paddle method.

FIG. 4 is a plot of the drug release profiles for various insulin formulations using data taken with the continuous flow-through cell.

FIG. 5 is a plot of the drug release profiles for an insulin formulation using data taken with the exemplary drug release cell of FIG. 1 having varying buffer concentrations in the liquid medium.

### DETAILED DESCRIPTION OF THE INVENTION

Disclosed herein is a drug release cell utilized in measuring the drug release rate of a product, such as a suspension or a drug dosage, in a liquid.

Formulations of drug dosages are often varied depending upon a variety of factors such as the desired length, release timing, and strength of the effect of the drug. One parameter that is important in obtaining the desired effects of the drug is the drug release rate of the drug dosage. The release of a drug dosage over time may be represented in the form of a drug release profile. Disclosed herein is a drug release cell that is useful in measuring the drug release rate of a drug dosage in a liquid.

Insulin is one such drug dosage that may have a variety of formulations. It is noted that while insulin is discussed as the product, the invention is not limited to insulin and may be used for testing of other liquid suspension drug products. The drug release cell may also be used for testing creams and gels. Insulin R (regular insulin) is an immediate release formulation and takes effect rapidly and has a relatively short duration of activity (4 to 12 hours). Insulin R has had nothing added to change the speed or length of action. Insulin N (NPH insulin) is an isophane suspension, which is a crystalline suspension of human insulin formed in the presence of zinc ions and protamine. Insulin N is utilized for extended release purposes and has a slower onset of action than insulin R and a longer duration of activity (up to 24 hours) than insulin R. Formulations that have onsets of action and duration of activity in between insulin R and insulin N may also be formed by mixing the two. For example, insulin 50/50 is 50% insulin N and 50% insulin R and insulin 70/30 is 70% insulin N and 30% insulin R.

The amount of drug released for different insulin formulations may be predicted utilizing the following formula:

(RX)ᵢ=(RN)ᵢ*(X/100)+(100−X)

(RX)ᵢ is the drug release at the time point (i) for the formulation of interest (X), in % release. (RN)ᵢ is the observed drug release for insulin N at the time point (i) in % release. X is the insulin formulation and is 70 for insulin 70/30, 50 for insulin 50/50 and 0 for insulin R. The drug release cell disclosed herein is useful in measuring the drug release rate of a variety of insulin formulations to determine if they match the predicted values. The drug release rates are in turn utilized to obtain distinguishable drug release profiles for each formulation of insulin.

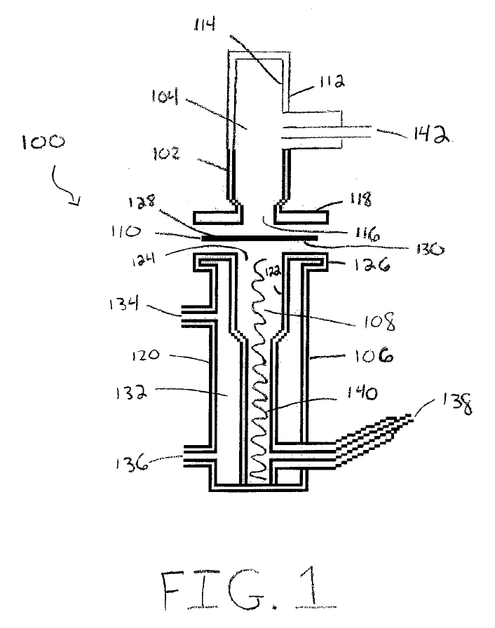

As shown in FIG. 1, drug release cell 100 has a first vessel 102 having a first chamber 104 and a second vessel 106 having a second chamber 108 and are separated by a membrane 110.

First vessel 102 may be generally cylindrical in shape with an exterior wall 112, an interior wall 114, an opening 116 leading to first chamber 104, and a flange 118 surrounding opening 116. Second vessel 106 may be similarly generally cylindrical in shape with an exterior wall 120, an interior wall 122, an opening 124, and a flange 126 surrounding opening 124. First vessel 102 and second vessel 106 may be made of an inert and non-absorbing material including, but not limited to pyrex.

Membrane 110 is sandwiched between first and second vessels 102, 106 such that a first surface 128 of membrane 110 is in contact with flange 118 of first vessel 102 and a second surface 130 of membrane 110 is in contact with flange 126 of second vessel 106. Flanges 118, 126 are clamped or otherwise held together in a liquid tight manner to maintain membrane 110 in place. Membrane 110 acts as a barrier between first chamber 104 and second chamber 108 preventing materials that are smaller than a pore size of the membrane from passing therethrough. Membrane 110 may have a pore size that prevents undissolved drug particles from passing through the membrane including, but is not limited to a pore size in a range from 0.1 μm to 1.0 μm. The material for membrane 110 may include, but is not limited to, polycarbonate, polyethersulfone, cellulose acetate, polyester, and nylon. Membrane 110 may be a 0.2 μm polyester.

Second vessel 106 may also have a water bath chamber 132 that surrounds second chamber 108 and has a water inlet 134 and a water outlet 136 such that water may constantly circulate through water bath chamber 132. The water bath may have a temperature of 37° C. to mimic human body temperature, but water baths at other temperatures may also be used. Second vessel 106 may also have a medium inlet 138 that leads to second chamber 108 and permits a liquid medium to be introduced to second chamber 108. A mixing device 140 is positioned in second chamber 108 to stir the liquid medium. Mixing device 140 may include, but is not limited to, a rotatable coil. Material for a rotatable coil may be inert and have sufficient mechanical strength, including but not limited to, stainless steel and may be sized to match the dimensions of second vessel 106. A magnetic stirrer may be attached to the bottom of the rotatable coil.

A product may be placed in second chamber 108 prior to flanges 118, 126 being clamped or otherwise held together in a liquid tight manner. Product may be a suspension of a drug dosage including, but not limited to, insulin. Subsequent to clamping flanges 118, 126 together the liquid medium is introduced into second chamber 108 through medium inlet 138. The liquid medium is able to pass through the pores in membrane 110 and fills first chamber 104 to at least above a sample port 142 connected to first chamber 104. Sample port 142 permits sampling of the liquid medium present in first chamber 104 to determine its contents utilizing high performance liquid chromatography (HPLC) or other analytical techniques.

Product, such as an insulin suspension, releases over time into the liquid medium. Mixing device 140 aids in dispersing the released product throughout the liquid medium. Media that has dissolved to a particle size smaller than the pore size of membrane 110 passes through membrane 110 into first chamber 104 and this movement through membrane 110 is also facilitated by the stirring or mixing motion of mixing device 140. The rate of release of product can be determined by utilizing sample port 142 to take samples of the liquid medium at selected time intervals and determine a concentration of product in the sampled portion.

New liquid medium is supplied through medium inlet 138 when samples are taken from sample port 142 in order to maintain the volume liquid medium in drug release cell 100. The liquid medium may have a pH in a range from about 6.5 to about 8.0. The liquid medium may be, but is not limited to, a buffer solution or a combination of buffer solution and water. The buffer solution may be a phosphate buffered saline (PBS). Introducing fresh liquid medium is also important when the product is an insulin suspension as it aids the drug release. Phosphate ions in the buffer solution are utilized to interact with the zinc ions in the crystallized insulin suspension to form a zinc phosphate derivative and consequently release the crystallized insulin. Accordingly, the amount of insulin released depends on the phosphate concentration.

Drug release cell 100 permits studies to find the drug release rates of different drug dosage formulations. This data may then be utilized to determine a drug release profile for formulation and to determine what effect varying the liquid medium or the drug dosage formulation, for example, the ratio of insulin N to insulin R has on the drug release profile. Testing with drug release cell 100 has the advantage of ensuring quality control and stability of the product.

### EXAMPLES

### Example 1

Suspensions of insulin N, 70/30, and 50/50 were separately tested in a drug release cell similar to that depicted in FIG. 1 having a 0.45 μm cellulose acetate membrane. Samples were taken at predetermined times and the amount of insulin released was determined as a percent of the labeled claim (% LC), which is the amount of insulin that should be released relative to the label amount on the bottle. The data was utilized to determine a drug release profile for each formulation by plotting % LC on the y-axis and time on the x-axis.

| Time | % | % | % |
|---|---|---|---|
| (min) | LC Insulin N | LC Insulin 70/30 | LC Insulin 50/50 |
| 2 | 0 | 30 | 51 |
| 12 | 12 | 43 | 58 |
| 22 | 36 | 66 | 74 |
| 32 | 85 | 97 | 99 |
| 42 | 101 | 104 | 106 |
| 52 | 100 | 100 | 100 |

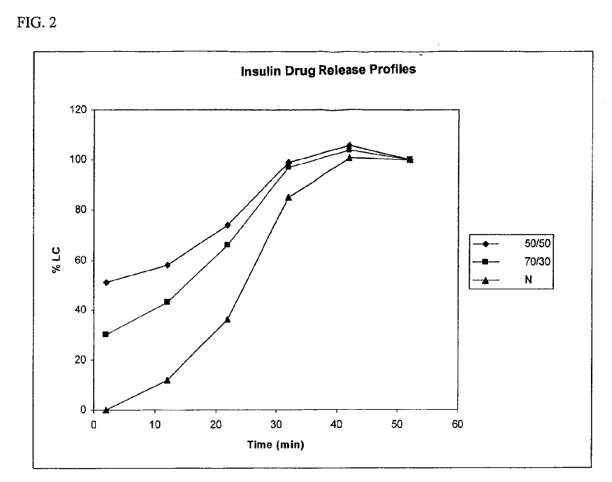

The drug release profile for each of the suspensions is shown in FIG. 2. As can be seen, each of the drug release profiles is distinguishable from one another.

### Example 2

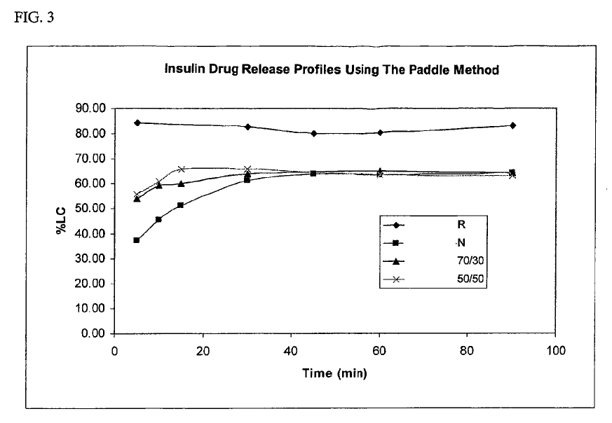

Amounts of Insulin R, N, 70/30, and 50/50 were separately tested in an apparatus similar to that described as the paddle method in the background section above. The liquid medium was 1 part PBS solution to 2 parts water and had a volume of 900 ml. The paddle was rotated at 50 rpm. Samples were taken at predetermined times and the amount of insulin released was determined as a percent of the labeled claim (% LC). The data was utilized to determine a drug release profile for each formulation by plotting % LC on the y-axis and time on the x-axis. The drug release profile for each insulin formulation is shown in FIG. 3. As can be seen in FIG. 3, the drug release profiles of insulin N, 70/30, and 50/50 are not easily distinguishable from one another, as is the case for the drug release profiles of the same formulations in FIG. 2 when utilizing a drug release cell similar to that depicted in FIG. 1. Further it is difficult to introduce fresh liquid medium in the paddle method, which is needed to fully release the insulin.

### Example 3

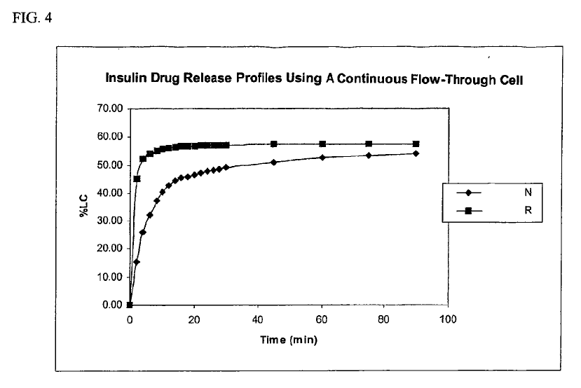

Amounts of Insulin R and N were separately tested in an apparatus similar to that described as the continuous flow-through cell in the background section above. The liquid medium was PBS solution and insulin formulations were tested at a flow rate of 4 ml/min. Samples were taken at predetermined times and the amount of insulin released was determined as a percent of the labeled claim (% LC). The data was utilized to determine a drug release profile for each formulation by plotting % LC on the y-axis and time on the x-axis. The drug release profile for each insulin formulation is shown in FIG. 4. As can be seen in FIG. 4, insulin R and N do not reach a drug release of 100% LC as a result of the sticking of particles to the filter. In addition, a continuous flow-through cell does not give reproducible results and requires many sampling points and samples.

### Example 4

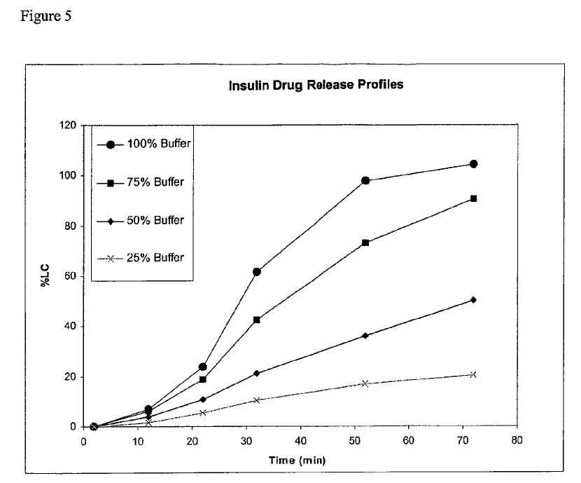

Crystalline suspensions of insulin N were separately tested in a drug release cell similar to that depicted in FIG. 1 in a liquid having concentrations of 25% buffer, 50% buffer, 75% buffer, and 100% buffer. The 100% buffer composition was 15 mM of sodium chloride, 0.34 mM of potassium chloride and 176 mM of phosphate. The 25% buffer, 50% buffer and 75% buffer were diluted with appropriate amounts of water. Samples were taken at predetermined times and the amount of insulin released was determined as a percent of the labeled claim (% LC). The data was utilized to determine a drug release profile for each formulation by plotting % LC on the y-axis and time on the x-axis. As shown in FIG. 5, the greater the buffer percentage the more rapid the release of the insulin.

As noted elsewhere, these example embodiments have been described for illustrative purposes only, and are not limiting. Other embodiments are possible and are covered by the methods and the drug release cell described herein. Such embodiments will be apparent to persons skilled in the relevant art(s) based on the teachings contained herein. Thus, the breadth and scope of the methods and the drug release cell described herein should not be limited by any of the above-described exemplary embodiments, but should be defined only in accordance with the following claims and their equivalents.

What is claimed is:

1. A drug release cell comprising:
a first vessel having a first chamber;
a second vessel having a second chamber;
a membrane positioned between said first vessel and said second vessel such that said first chamber is located above said membrane and said second chamber is located below said membrane, wherein said membrane has pores sized to permit a liquid and a released product disposed in said second chamber to pass therethrough;
a sample port connected to said first chamber; and
a mixing device located in said second chamber.

2. The drug release cell of claim 1, wherein said mixing device is a rotatable coil.

3. The drug release cell of claim 1, further comprising a refilling port connected to said second chamber.

4. The drug release cell of claim 1, wherein said second vessel further comprises:
a water bath chamber surrounding said second chamber;
a water inlet connected to said water bath chamber; and
a water outlet connected to said water bath chamber.

5. A method for testing the release of a product in a liquid, comprising the steps of:
providing a drug release cell comprising:
a first vessel having a first chamber;
a second vessel having a second chamber;
a membrane positioned between said first vessel and said second vessel such that said first chamber is located above said membrane and said second chamber is located below said membrane, wherein said membrane has pores sized to permit said liquid disposed in said second chamber and said product released in said liquid to pass therethrough;
a sample port connected to said first chamber; and
a mixing device located in said second chamber;
placing said product in said second chamber;
filling said drug release cell with said liquid; and
mixing said liquid utilizing said mixing device to circulate said liquid and evenly disperse said drug throughout said liquid as it releases.

6. The method of claim 5, wherein said product is a suspension.

7. The method of claim 5, wherein said product is a drug dosage.

8. The method of claim 5, wherein said product is a suspension of insulin.

9. The method of claim 5, wherein said filling step comprises filling said drug release cell with said liquid such that said liquid fills said second chamber and at least a portion of said first chamber.

10. The method of claim 5, further comprising sampling a quantity of said liquid in said first chamber utilizing said sample port at selected time intervals to determine a concentration of said product released in said liquid.

11. The method of claim 5, wherein said liquid is a phosphate buffered saline.

12. The method of claim 5, wherein said mixing device is a rotatable coil.

13. A method for testing the drug release of a suspension in a liquid, comprising the steps of:
providing a drug release cell comprising:
a first vessel having a first chamber;
a second vessel having a second chamber; and
a membrane positioned between said first vessel and said second vessel such that said first chamber is located above said membrane and said second chamber is located below said membrane, wherein said membrane has pores sized to permit said liquid disposed in said second chamber and said suspension released in said liquid to pass therethrough;
placing said suspension in said second chamber; and
filling said drug release cell with said liquid;
wherein said drug release cell gives data that can be utilized to determine distinguishable drug release profiles for suspensions having different rates of release.

14. The method of claim 13, wherein said suspension is a drug dosage.

15. The method of claim 14, wherein said drug dosage is insulin.

16. The method of claim 14, wherein said drug release cell gives distinguishable drug release profiles for drug dosages of the same drug having different rates of release.

17. The method of claim 14, wherein said drug release cell gives distinguishable drug release profiles for different formulations of said drug dosage, each having different rates of release.

18. The method of claim 13, wherein said liquid is a phosphate buffered saline.
